(19) United States
(12) Patent Application Publication
(10) Pub. No.: US 2025/0367226 A1
(43) Pub. Date: Dec. 4, 2025

(54) ANTI-MALASSEZIA AGENT

(71) Applicant: N.T.H Co., Ltd., Tokyo (JP)

(72) Inventors: Yoshimi ODA, Tokyo (JP); Souichi NAKASHIMA, Kyoto (JP)

(73) Assignee: N.T.H Co., Ltd., Tokyo (JP)

(21) Appl. No.: 19/223,384

(22) Filed: May 30, 2025

(30) Foreign Application Priority Data
Jun. 3, 2024 (JP) 2024-090154

(51) Int. Cl. A61K 31/7034; A61K 31/7048; A61K 36/185; A61P 31/10; C07H 15/203

(52) U.S. Cl. CPC A61K 31/7034; A61K 31/7048; A61K 36/185; A61P 31/10; C07H 15/203

(57) ABSTRACT

Problem to be Solved: To provide an anti-Malassezia agent and a method for producing an active ingredient of the anti-Malassezia agent.

Solution to the Problem: Provided is an anti-Malassezia agent comprising any one of compounds (I), (II) and (III) represented by the following formulas (I), (II) and (III):

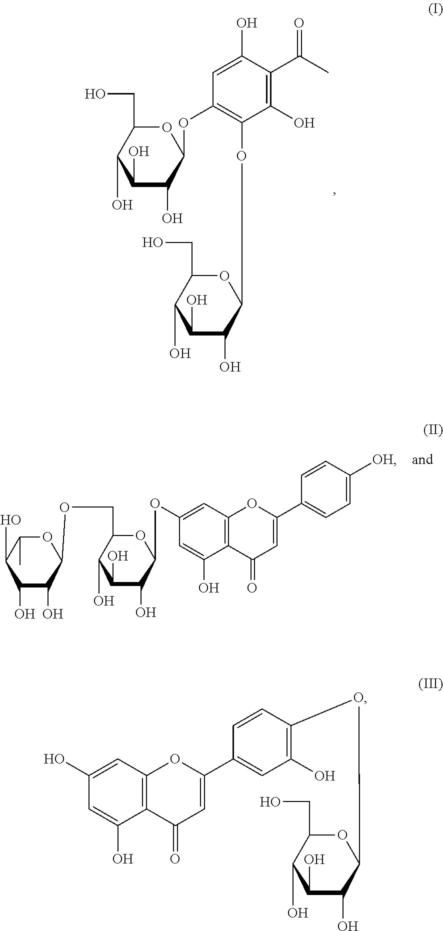

respectively, or a pharmaceutically acceptable salt thereof, or a henna extract. A method for producing the compound (I) is also provided.

Selected Figure: none.

### TECHNICAL FIELD

The present invention relates to an anti-Malassezia agent.

### BACKGROUND ART

Malassezia is known as one of scalp-resident fungi associated with dandruff and seborrheic dermatitis. Malassezia degrades sebum to release fatty acids. Excessive proliferation of Malassezia has been reported to be responsible for dandruff, folliculitis or dermatitis. The balance of various bacteria and fungi (including Malassezia) on the scalp is said to be important, but the details have yet to be fully clarified. For example, antifungal agents are widely used for the improvement of dandruff, but many cases cannot be completely cured with antifungal agents. Malassezia has recently been reported to also affect atopy.

Henna (Lawsonia inermis) (also called hina, mignonette tree, or shikoka in Japanese) is a woody plant belonging to the genus Lawsonia of the family Lythraceae. Lawsone (2-hydroxy-1,4-naphthoquinone) is the characteristic component of the plant and has been reported to have various pharmacological activities, including an antioxidant effect and antibacterial and antifungal effects (Patent Literature 1).

Patent Literature 2 describes that the compound represented by the formula below was isolated from an extract of flowers of henna. The literature also describes that the compound exhibits an enhancing effect on the differentiation of neuron-like cells and is useful for preventing or treating neurological diseases.

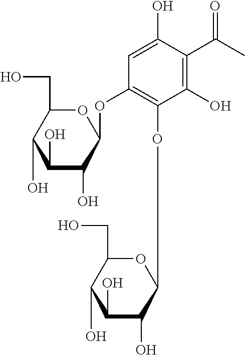

### CITATION LIST

### Patent Literature

Patent Literature 1: JP 2024-7027 A

Patent Literature 2: JP pat. No. 5728105

### SUMMARY OF INVENTION

### Technical Problem

An object of the present invention is to provide an anti-Malassezia agent and a method for producing an active ingredient of the anti-Malassezia agent.

### Solution to Problem

The present invention was made to solve the above problems and includes the following.
[1] An anti-Malassezia agent comprising a compound (I) represented by the following formula (I):

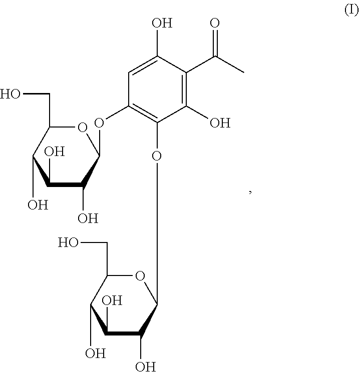

or
a pharmaceutically acceptable salt thereof.
[2] An anti-Malassezia agent comprising a compound (II) represented by the following formula (II):

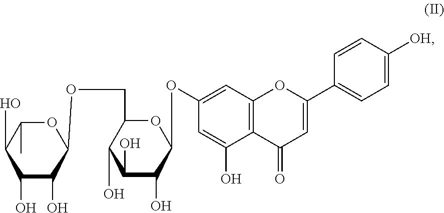

or
a pharmaceutically acceptable salt thereof.
[3] An anti-Malassezia agent comprising a compound (III) represented by the following formula (III):

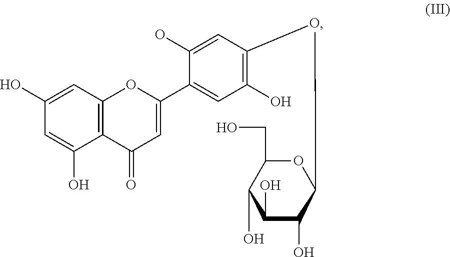

or
a pharmaceutically acceptable salt thereof.
[4] An anti-Malassezia agent comprising a henna extract.
[5] The anti-Malassezia agent according to the above [4], wherein the henna extract comprises at least one of compounds (I), (II) and (III) represented by the following formulas (I), (II) and (III):

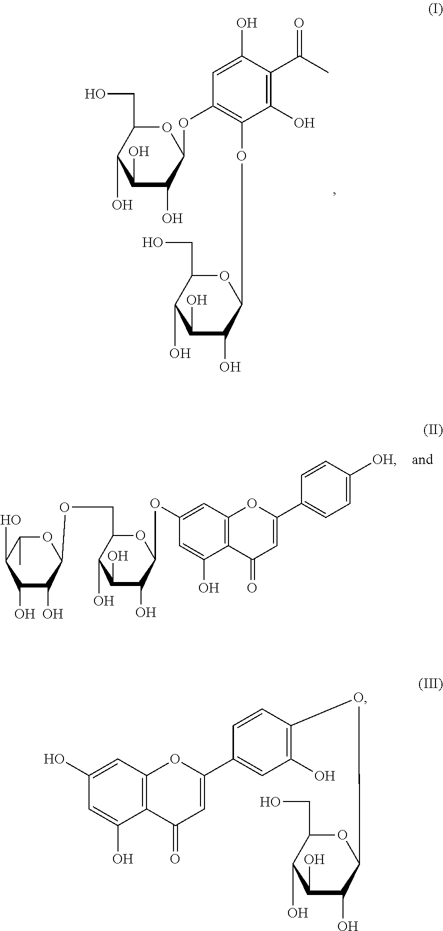

respectively, or a pharmaceutically acceptable salt thereof.
[6] The anti-Malassezia agent according to the above [4], wherein the henna extract is a henna leaf extract.
[7] A method for producing a compound (I) represented by the following formula (I):

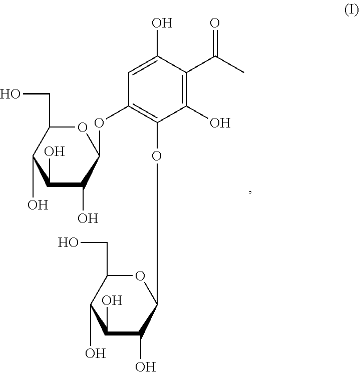

or
a pharmaceutically acceptable salt thereof, comprising
   subjecting leaves of henna to extraction to prepare an extract, and
   obtaining the compound from the extract.

### Advantageous Effects of Invention

The present invention provides an anti-Malassezia agent. The anti-Malassezia agent is useful for use in pharmaceutical compositions and cosmetics. The present invention also provides a novel method for producing a compound (I) that can be used as an active ingredient of the anti-Malassezia agent.

### BRIEF DESCRIPTION OF DRAWINGS

FIG. 1 is a chart showing the process of the preparation of a henna leaf extract and the isolation of compounds 1 to 3.

FIG. 2 is a graph showing the results of anti-Malassezia tests (the viability (%) of Malassezia) using a henna leaf extract.

FIG. 3 is a graph showing the results of anti-Malassezia tests (the viability (%) of Malassezia) using compound 1.

FIG. 4 is a graph showing the results of anti-Malassezia tests (the viability (%) of Malassezia) using compound 2.

FIG. 5 is a graph showing the results of anti-Malassezia tests (the viability (%) of Malassezia) using compound 3.

### DESCRIPTION OF EMBODIMENTS

### Anti-Malassezia Agent

The present invention provides an anti-Malassezia agent. Malassezia is yeast-like fungi that reside on the surface of the skin of mammals including humans. Malassezia has yeast and hyphae forms in its life cycle. Malassezia is used without distinguishing the two distinct forms in the present invention. Malassezia requires lipids for their growth. Malassezia includes Malassezia globosa, Malassezia furfur, Malassezia restricta, Malassezia obtusa, and other species. The term “anti-Malassezia” effect as used herein refers to the effect of inhibiting the proliferation of Malassezia, in particular, in the environment that Malassezia favors (e.g., an environment where lipids such as sebum are present). The anti-Malassezia effect can be examined by investigating the growth of Malassezia in a medium containing a lipid component. The anti-Malassezia effect can also be examined by, for example, the testing method in Example 2 described later.

The anti-Malassezia agent of the present invention comprises any one of the compounds described later or a henna extract. The compounds and the henna extract may be collectively called herein the “active ingredient of the anti-Malassezia agent.”

The active ingredient of the anti-Malassezia agent of the present invention can be obtained or purified by separation through preparation of an extract from henna or any combination of treatments selected from extraction from a henna extract by liquid separation using a solvent; fractionation by chromatography in various modes of separation (e.g., ion exchange, hydrophilic adsorption, hydrophobic adsorption, size exclusion, ligand exchange, affinity, etc.); molecular weight fractionation by filtration using a filter paper, a membrane filter or an ultrafiltration membrane; application of pressure or reduced pressure; heating or cooling; drying; pH adjustment; deodorization; depigmentation; long-term static storage; etc. For example, a compound used as the active ingredient can be isolated by a combination of column chromatography and preparative HPLC in accordance with the method in Example 1 described later. The isolated compound, if necessary, can be subjected to structural determination by NMR or mass spectrography. The compound as the active ingredient can be used in the isolated form, or may be in the form contained in a henna extract.

The active ingredient of the anti-Malassezia agent of the present invention will be described below.

### 1. Compounds

In an aspect of the present invention, the active ingredient of the anti-Malassezia agent of the present invention is any one of the compounds described below. The structural formulas of the compounds as the active ingredient of the present invention are as follows:

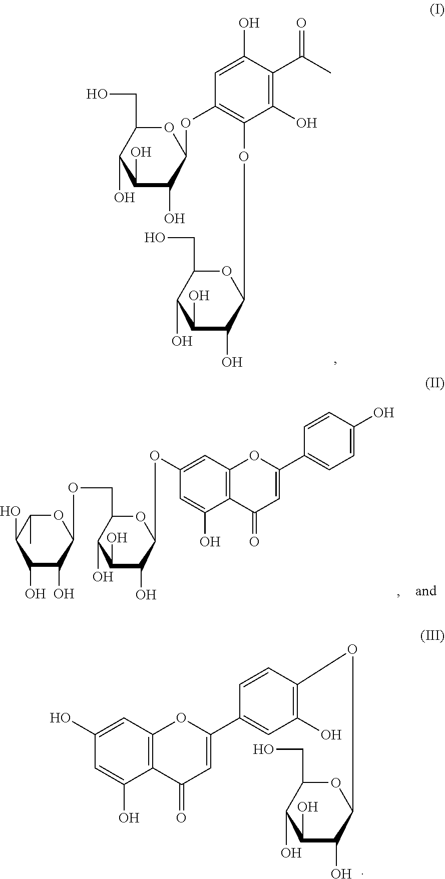

The above formulas (I), (II) and (III) are the structural formulas of inermioside A (compound 1), apigenin 7-O-α-L-rhamnopyranosyl-(1→6)-β-D-glucopyranoside (compound 2) and luteolin 4′-O-β-D-glucopyranoside (compound 3), respectively. The compounds represented by the formulas are called compounds (I), (II) and (III), respectively. These compounds are collectively called the “compound of the present invention.”

The compound of the present invention includes stereoisomers, optical isomers, and a mixture of these isomers; and solvates, polymorphic forms, and isotopically labeled compounds.

The compound of the present invention may have a chiral carbon atom. In such a case, the compound of the present invention may exist as a stereoisomer. The present invention includes any optical isomers, for example, stereoisomers of the compounds of the formulas (I), (II) and (III) (compounds 1, 2 and 3) (including enantiomers, diastereomers, and a mixture thereof (e.g., racemates)). Various stereoisomers may be separated or resolved from each other by a conventional method, or an isomer may be obtained by a conventional stereoselective synthetic method or a conventional asymmetric synthetic method.

The compound of the present invention may be present as various tautomers, and the present invention is understood to include such all tautomers.

The compound of the present invention may be produced in a crystalline form or an amorphous form. When the compound of the present invention is in a crystalline form, the compound may be hydrated or solvated. The present invention includes, in its scope, stoichiometric hydrates or solvates, and compounds containing a variable amount of water and/or a solvent.

The compound of the present invention may have polymorphic forms, which are also included in the scope of the present invention.

The present invention also includes isotopically labeled compounds (the isotopically labeled compounds are the same as the compounds described herein except that one or more atoms are replaced with an atom with an atomic mass or a mass number that is different from the atomic mass or the mass number usually found in the nature). The isotopes that may be incorporated into the compound of the present invention may be the isotopes of hydrogen, carbon, nitrogen, oxygen, phosphorus, fluorine, iodine, or chlorine, for example, ³H, ¹¹C, ¹⁴C, ¹⁸F, ¹²³I, and ¹²⁵I. The compound of the present invention containing such an isotope and/or the isotopes of other atoms are within the scope of the present invention.

The compound of the present invention also includes ester derivatives and ether derivatives. It will be readily apparent to a person skilled in the art that when a glycoside has bioactivity, an ester or ether derivative of the glycoside also has a similar effect. For example, when an ester or ether derivative is administered to a body or a body surface, the ester or ether derivative can be hydrolyzed by an enzyme or an acid under physiological conditions and converted into any one of the compounds of the formulas (compounds 1, 2 and 3) to exhibit the desired activity. Examples of the ester include C₁-C₆ alkyl esters, which have an alkyl group, such as methyl esters and ethyl esters. Examples of the ether include C₁-C₆ alkyl ethers, which have a C₁-C₆ alkyl group, such as methyl ethers and ethyl ethers.

The “alkyl group” as used herein may have a branch, a cyclic structure, and/or an unsaturated bond. The term “C₁-C₆” in a C₁-C₆ alkyl group is defined to refer to a linear or branched group of 1, 2, 3, 4, 5 or 6 carbon atoms. Specific examples of a C₁-C₆ alkyl group thus include, but are not limited to, a methyl group, an ethyl group, an ethenyl group, a Z-ethylene group, an n-propyl group, an iso-propyl group, a cyclopropyl group, a propenyl group, an n-butyl group, an iso-butyl group, a tert-butyl group, a 1-butenyl group, a pentyl group, a hexyl group, and a cyclohexyl group.

Esters and ethers can be produced using a commercially available reagent or a known method. For example, an ester derivative can be produced by the Fischer esterification reaction using a catalyst such as sulfuric acid. In other words, an ester derivative of the compound of the present invention can be obtained by dehydration condensation of a hydroxy group in the formula (I), (II) or (III) with an oxoacid such as carboxylic acid, sulfuric acid, phosphoric acid, or nitric acid. An ester can also be produced by the Schotten-Baumann reaction using an acid anhydride such as acetic anhydride or an acid halide. An ether derivative is produced by dehydration condensation of a hydroxy group in the formula (I), (II) or (III) and an alcohol. An ether can also be produced by reacting a sodium alkoxide formed from a hydroxy group in the formula (I), (II) or (III) with an alkyl halide. The Williamson ether synthesis method can be used for an ether synthesis reaction by alkylating a metal alcoholate formed from a hydroxy group in the formula (I), (II) or (III) with a sulfuric ester, or a reaction by alkylating an alcohol with an alkyl halide in the presence of a tertiary amine.

When the compound of the present invention has a basic group, the compound can form an acid addition salt. Examples of such a salt include acid addition salts formed with an inorganic acid, such as hydrochloric acid, hydrobromic acid, sulfuric acid, nitric acid, or phosphoric acid; and acid addition salts formed with an organic acid, such as succinic acid, maleic acid, formic acid, acetic acid, trifluoroacetic acid, propionic acid, fumaric acid, citric acid, tartaric acid, benzoic acid, p-toluenesulfonic acid, methanesulfonic acid, or naphthalenesulfonic acid. When the compound herein contains an acidic functional group such as a carboxy group, the counter ions to the acidic functional group include not only organic bases but also inorganic salts selected from sodium salts, potassium salts, lithium salts, calcium salts, magnesium salts, etc. These salts should be pharmaceutically acceptable. The compound of the present invention may include a compound that can form an acid addition salt with 1 equivalent or more of the above acid. The present invention includes all possible stoichiometric forms and non-stoichiometric forms in its scope.

A pharmaceutically acceptable salt of the compound of the present invention is also used as an active ingredient of the anti-Malassezia agent. The pharmaceutically acceptable salt may be any salt that retains the efficacy of the active ingredient and does not have an adverse effect on human bodies. Examples of the pharmaceutically acceptable salt include salts with an acid, such as acetic acid, propionic acid, butyric acid, formic acid, trifluoroacetic acid, maleic acid, tartaric acid, citric acid, stearic acid, succinic acid, ethylsuccinic acid, malonic acid, lactobionic acid, gluconic acid, glucoheptonic acid, benzoic acid, methanesulfonic acid, ethanesulfonic acid, 2-hydroxyethanesulfonic acid, benzenesulfonic acid, p-toluenesulfonic acid (tosylic acid), lauryl sulfate, malic acid, aspartic acid, glutamic acid, adipic acid, cysteine, N-acetylcysteine, hydrochloric acid, hydrobromic acid, phosphoric acid, sulfuric acid, hydroiodic acid, nicotinic acid, oxalic acid, picric acid, thiocyanic acid, undecanoic acid, an acrylic acid polymer, or a carboxyvinyl polymer; salts with an inorganic base, such as a lithium salt, a sodium salt, a potassium salt, or a calcium salt; salts with an organic amine, such as morpholine or piperidine; salts with an amino acid; etc.

### 2. Henna Extract

In another aspect of the present invention, the active ingredient of the anti-Malassezia agent of the present invention is a henna extract. Henna (Lawsonia inermis) (also called hina, mignonette tree, or shikoka in Japanese) is a woody plant belonging to the genus Lawsonia of the family Lythraceae. Henna grows on dry hill areas with good drainage. The growing regions of henna are Egypt, India, North Africa, and Iran, among others, but may be any region where henna can grow. Henna is an evergreen shrub or small tree of about 3 to 6 m in height. Henna has small four-petal flowers in white or pink, measuring about 7 mm in diameter, and oval or lanceolate leaves of about 2 to 4 cm in length and about 1 to 2 cm in width. The henna extract can be prepared as described later.

In one embodiment, the henna extract as the active ingredient of the anti-Malassezia agent of the present invention is a henna extract containing the compound of the present invention or a pharmaceutically acceptable salt thereof. In other words, the henna extract as the active ingredient contains at least one of compounds (I), (II) and (III) represented by the following formulas (I), (II) and (III), respectively, or a pharmaceutically acceptable salt thereof. The details of the compounds are as described above.

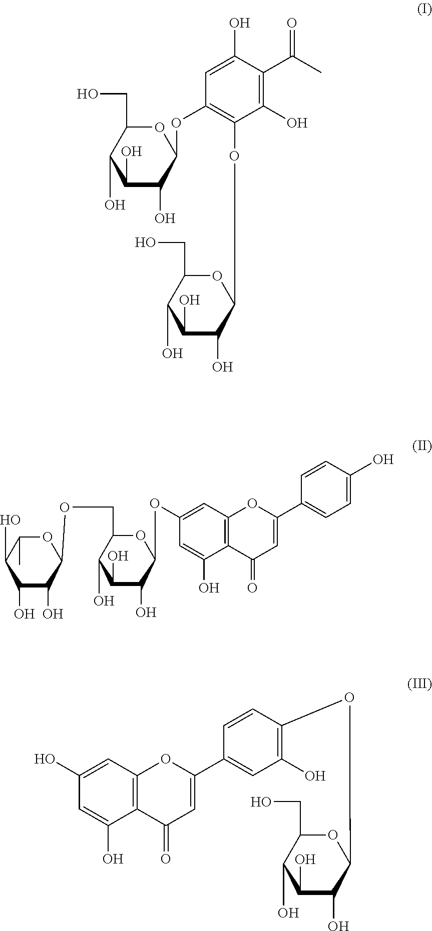

Chromatography fractionation of a henna leaf extract revealed that a henna leaf extract contains the compounds of the above formulas (I), (II) and (III) as described later, which are inermioside A (compound 1), apigenin 7-O-α-L-rhamnopyranosyl-(1→6)-β-D-glucopyranoside (compound 2) and luteolin 4′-O-β-D-glucopyranoside (compound 3), respectively. The compound (I) is also found in an extract from flowers of henna (Patent Literature 2).

The henna extract can be obtained by subjecting a plant body of henna or its part to extraction. For example, the henna extract can be obtained by drying a plant body of henna or its part, and subjecting the dried plant body or its part to extraction with a solvent. The growing regions and the degree of growth of the plant body of henna used for extraction are not limited to particular ones. The part of henna used for extraction may be the leaves or flowers of henna, or both, and is preferably the leaves of henna. In one embodiment, the henna extract is a henna leaf extract. The drying is not limited to a particular method, and may be, for example, lyophilization, spray drying, etc. The extraction solvent may be, but is not limited to, for example, an alcohol such as methanol or ethanol, or a solvent commonly used for the production of cosmetics, for example, butylene glycol (BG), propylene glycol (PG), or 1,3-propanediol, and is preferably methanol. The extraction temperature may be any temperature that is usually employed for henna extraction, but the extraction may be, for example, heating extraction (at about 80° C.). The extraction time may be any length of time that allows the extraction of the active ingredient, and may be selected depending on the extraction temperature. If necessary, operations such as pulverization, filtration, re-extraction, and/or solvent evaporation may be performed. The henna extract may be concentrated by lyophilization, spray drying, or other methods. The henna extract may be in the form of a liquid or a powder.

### Production Method of Compound (I)

The present invention also provides a method for producing the compound (I) or a pharmaceutically acceptable salt thereof. The production method of the present invention comprises subjecting henna leaves to extraction to prepare an extract, and obtaining the compound from the extract. The compound (I) was newly found in flowers of henna for the first time (Patent Literature 2), but the present inventors found that the compound (I) is also produced from the leaves of henna. Flowers of henna used as a material for extraction are small buds with a size of about 1 mm, and leaves are much more abundant than flowers in the plant body of henna. Hence, according to the production method of the present invention, the compound (I), which was newly found in henna flowers, can be produced in a larger quantity than the production using henna flowers. The production method of the present invention is advantageous in that the compound can be produced in a large quantity at a low cost, and is also economically valuable.

The extraction method from henna leaves is as described in the above section “2. Henna extract.” The compound (I) can be obtained from the extract. To obtain the compound (I) from the extract, the compound of the formula (I) is isolated from the henna leaf extract, or alternatively, if desired, the henna leaf extract is subjected to an etherification reaction or an esterification reaction without the isolation of the compound. The isolation of the compound can be done by performing any combination of treatments selected from extraction from a henna extract by liquid separation using a solvent; fractionation by chromatography in various modes of separation (e.g., ion exchange, hydrophilic adsorption, hydrophobic adsorption, size exclusion, ligand exchange, affinity, etc.); molecular weight fractionation by filtration using a filter paper, a membrane filter or an ultrafiltration membrane; application of pressure or reduced pressure; heating or cooling; drying; pH adjustment; deodorization; depigmentation; long-term static storage; etc. For example, the isolation of the compound can be performed by the method in Example 1 described later. The etherification reaction and the esterification reaction are described in the above section “1. Compounds.”

### Applications

Malassezia is yeast-like fungi that reside on the surface of the skin of mammals including humans. Malassezia usually resides as resident fungi in the normal flora of the skin. Malassezia is responsible for diseases such as Malassezia folliculitis, Malassezia intertrigo, seborrheic dermatitis, dandruff, and pityriasis versicolor. Malassezia is also considered as an aggravating factor of atopic dermatitis. When Malassezia abnormally proliferates on the scalp, excessive amounts of fatty acids are produced, which may adversely affect the scalp and may cause scalp troubles. The anti-Malassezia agent of the present invention has an anti-Malassezia effect and is useful for preventing or treating diseases associated with Malassezia or for preventing or inhibiting harmful effects that may be caused by abnormal proliferation of Malassezia.

The anti-Malassezia agent of the present invention can be orally or parenterally applied, and may be incorporated in a product or a composition that is desired to have an anti-Malassezia effect. Examples of such a product or a composition may include medicaments (pharmaceutical compositions), quasi-drugs, cosmetics, and functional foods. Such a product and a composition are also within the scope of the present invention.

The product or composition of the present invention can be formulated by a conventional method, but are not limited thereto.

The dosage form of the product or composition may be, for example, topical agents and oral drugs. Examples of the topical agents include liniments, patches and sprays. Examples of the liniments include ointments, creams, solutions, gels, lotions, stick-type hard gels, etc. Examples of the patches include cataplasms, plasters, tapes, adhesive patches, etc. Examples of the sprays include aerosols. Examples of oral drugs include tablets, powders, fine granules, granules, coated tablets, capsules, syrups, troches, etc. For the formulation of these dosage forms, commonly used additives, such as an excipient, a binder, a lubricant, a colorant, and/or a flavor improver can be used, and if necessary, a stabilizer, an emulsifier, an absorption enhancer, a surfactant, a pH adjuster, an antiseptic, an antioxidant, etc. can also be used. Such a dosage form can be formulated by blending ingredients commonly used as materials for medicaments, quasi-drugs, cosmetics, functional foods, and the like according to a conventional method.

The anti-Malassezia agent of the present invention can also be used as cosmetics or quasi-drugs for topical application. Specific examples of such cosmetics or quasi-drugs include, but are not limited to, hair cosmetics, shampoos, rinses, conditioners, hair growers, scalp care products, skin care products, facial toners, facial oils, milky lotions, facial essences, creams, foundations, facial packs, cleansing creams or oils, facial cleansers, all-in-one gels, body lotions, etc. The anti-Malassezia agent of the present invention may be in the form of a food or drink for oral application. For example, the anti-Malassezia agent of the present invention may be used as a functional food (a health food) such as a food for special health use, a food with function claims, or a food with nutrient function claims. The anti-Malassezia agent of the present invention may be blended into a general food or drink.

Examples of ingredients for the medicaments, the quasi-drugs, the cosmetics, the functional foods, or the like include animal and vegetable oils, such as soybean oil, beef tallow, and synthetic glycerides; hydrocarbons, such as liquid paraffin, squalane, and solid paraffin; ester oils, such as octyldodecyl myristate and isopropyl myristate; higher alcohols, such as cetostearyl alcohol and behenyl alcohol; silicone resins; silicone oils; surfactants, such as polyoxyethylene fatty acid esters, sorbitan fatty acid esters, glycerol fatty acid esters, polyoxyethylene sorbitan fatty acid esters, polyoxyethylene hardened castor oils, and polyoxyethylene-polyoxypropylene block copolymers; water-soluble polymers, such as hydroxyethyl cellulose, polyacrylic acid, carboxyvinyl polymers, polyethylene glycol, polyvinylpyrrolidone, and methyl cellulose; lower alcohols, such as ethanol and isopropanol; polyhydric alcohols, such as glycerol, propylene glycol, dipropylene glycol and sorbitol; sugars, such as glucose and sucrose; inorganic powders, such as anhydrous silicic acid, magnesium aluminum silicate and aluminium silicate; purified water; etc. Examples of excipients include water, purified water, alcohols, glycerol, lactose, corn starch, sucrose, glucose, mannitol, sorbitol, crystalline cellulose, silicon dioxide, etc. Examples of binders include polyvinyl alcohol, polyvinyl ether, methyl cellulose, ethyl cellulose, gum arabic, tragacanth, gelatin, shellac, hydroxypropyl methylcellulose, hydroxypropyl cellulose, polyvinylpyrrolidone, polypropylene glycol-polyoxyethylene block polymers, meglumine, etc. Examples of disintegrants include starch, agar, gelatin powder, crystalline cellulose, calcium carbonate, sodium hydrogen carbonate, calcium citrate, dextrin, pectin, carboxymethylcellulose calcium, etc. Examples of lubricants include magnesium stearate, talc, polyethylene glycol, silica, hydrogenated vegetable oils, etc. Examples of colorants include those approved for addition to medicaments or cosmetics. Examples of flavor improvers include cocoa powder, menthol, aromatic powder, mentha oil, borneol, cinnamon powder, etc.

For example, the production method of the topical agent is not limited to a particular one, and the topical agent can be produced by a conventional method. The oral formulation is produced by, for example, combining the compound of the present invention or a derivative thereof or a hydrate thereof as an active ingredient with an excipient, if necessary, further adding, for example, a binder, a disintegrant, a lubricant, a colorant, a flavor improver, or the like, and formulating the mixture into, for example, a powder, fine granules, granules, a tablet, a coated tablet, a capsule, or the like by a conventional method. Needless to say, the tablet and the granules may be coated with, for example, sugar coating or other types of coating as appropriate for needs. The syrup and an injectable formulation are formulated through a conventional method by adding, for example, a pH modifier, a solubilizer, an isotonic agent, or the like, and if necessary, a solubilizing agent, a stabilizer, or the like. The base ingredient used may be any one of various ingredients that are commonly used in medicaments, quasi- drugs, cosmetics, functional foods, etc. Examples of the base ingredient include ingredients such as animal and vegetable oils, mineral oils, ester oils, waxes, higher alcohols, fatty acids, silicone oils, surfactants, phospholipids, alcohols, polyhydric alcohols, water-soluble polymers, clay minerals, purified water, etc. If necessary, a pH modifier, an antioxidant, a chelating agent, an antibacterial and antifungal agent, a colorant, a fragrance, etc. can also be added. Further, if necessary, other ingredients, such as a blood flow increasing agent, a bactericide, an anti-inflammatory, a cell activator, a vitamin, an amino acid, a moisturizer, or a keratolytic agent can also be blended.

The amount of the active ingredient in the anti-Malassezia agent of the present invention contained in the product or composition of the present invention is not limited to a particular amount, and is adjusted as appropriate for the purpose. For example, the amount used is an adequate dose to exhibit the anti-Malassezia effect. The amount of the active ingredient of the anti-Malassezia agent of the present invention contained in the formulation of the pharmaceutical composition may vary with various factors such as the form of the formulation. The amount of the compound or a pharmaceutically acceptable salt thereof may be, for example, 0.001% by mass or more, 0.005% by mass or more, or 0.01% by mass or more, and may be, for example, 30% by mass or less, 20% by mass or less, or 10% by mass or less relative to the total amount of the formulation.

The dosage of the pharmaceutical composition of the present invention may vary with various factors, such as the severity of the symptoms, the age, sex and body weight of the subject, the dosage form, the type of salt and the specific disease. The compound or a pharmaceutically acceptable salt thereof as the active ingredient of the anti-Malassezia agent of the present invention may typically be administered to a human adult at a dose of about 30 μg to 10 g, preferably 100 μg to 5 g, more preferably 100 μg to 100 mg a day as a single dose or several divided doses.

The amount of the active ingredient of the anti-Malassezia agent of the present invention contained in the quasi-drug, the cosmetic or the functional food of the present invention and the frequency of use thereof may be in accordance with those of the pharmaceutical composition of the present invention and may be adjusted as appropriate for the purpose.

The anti-Malassezia agent of the present invention may be used together with one or more compounds that are known to be useful for treating or preventing skin diseases or their symptoms associated with Malassezia.

The present invention will be described in more detail below with reference to Examples, but the present invention is not limited thereto.

### EXAMPLES

The present invention will be described in more detail below with reference to specific Examples, but the present invention is not limited thereto. The compounds, reagents, etc. used in Examples to carry out the present invention may be easily obtained as commercially available products or can be synthesized by methods known in the art, unless otherwise specified.

### Example 1: Preparation of Henna Leaf Extract and Isolation of Compounds

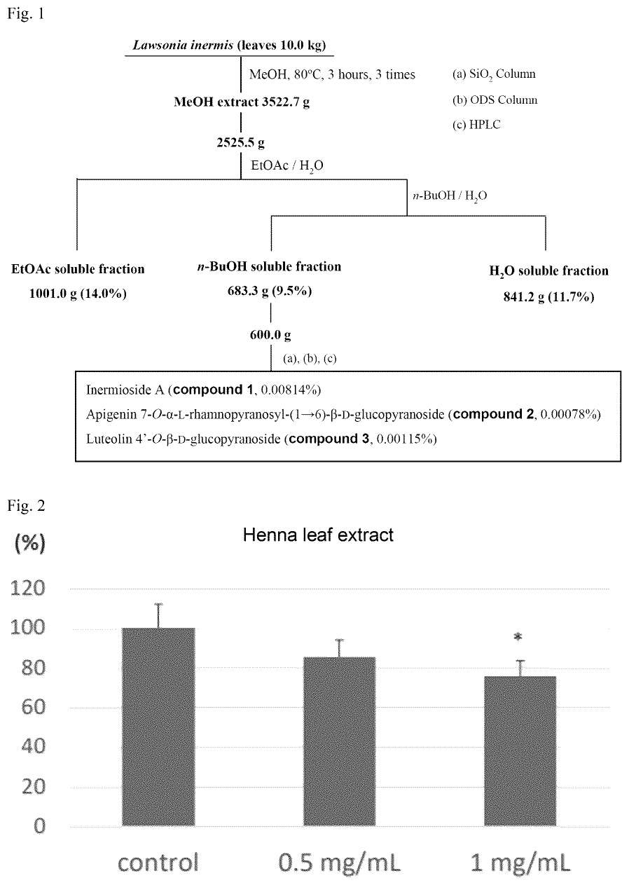

A henna leaf extract was prepared and compounds were isolated from the henna leaf extract by the procedure shown in FIG. 1. Details of the procedure will be described below.

Dried leaves (10.0 kg) of henna (Lawsonia inermis) made in Kyoto were subjected to extraction with methanol (MeOH) under heating (at 80° C.). The extraction liquid was filtered and MeOH was added to the residue. This extraction procedure was repeated three times. The liquids obtained by the MeOH extraction were combined and the solvent was evaporated in vacuo to give a MeOH extract (3522.7 g, 35.2% yield). (this extract was used as a henna leaf extract in Example 2 described later).

An aliquot of 2525.5 g of the obtained MeOH extract was subjected to partition extraction with ethyl acetate (EtOAc) and water (H₂O). The resulting H₂O soluble fraction was further subjected to partition extraction with n-butanol (BuOH). The solvents of the soluble fractions were evaporated in vacuo to give an EtOAc soluble fraction (1001.0 g, 14.0% yield), an n-BuOH soluble fraction (683.3 g, 9.5% yield) and an H₂O soluble fraction (841.2 g, 11.7% yield).

The n-BuOH soluble fraction (600.0 g) obtained above was fractionated by normal phase silica gel column chromatography [3.0 kg, CHCl₃:MeOH:H₂O=(30:3:1)→(20:3:1)→(10:3:1)→(7:3:1)→(6:4:1)→(5:5:1)→MeOH] to give the following fractions: Fr. B1 (5.3 g), Fr. B2 (22.2 g), Fr. B3 (50.3 g), Fr. B4 (256.9 g), Fr. B5 (182.6 g) and Fr. B6 (3.5 g). (Here, Fr. B1 denotes the first fraction, Fr. B2 denotes the second fraction, Fr. B3 denotes the third fraction, Fr. B4 denotes the fourth fraction, Fr. B5 denotes the fifth fraction, and Fr. B6 denotes the sixth fraction obtained by fractionating the n-BuOH soluble fraction. A fraction derived from each fraction is indicated by a hyphen. For example, Fr. B5-6-4 denotes the fourth fraction obtained by fractionating the sixth fraction obtained by fractionating the fifth fraction of the n-BuOH soluble fraction.)

Fr. B5 (182.6 g) was fractionated by normal phase silica gel column chromatography [1.5 kg, CHCl₃:MeOH=(7:1)→(5:1)→(3:1)→(2:1)→(1:1)→MeOH] to give the following fractions: Fr. B5-1 (1.8 g), Fr. B5-2 (1.6 g), Fr. B5-3 (6.5 g), Fr. B5-4 (5.9 g), Fr. B5-5 (17.9 g), Fr. B5-6 (36.2 g), Fr. B5-7 (27.5 g), Fr. B5-8 (34.3 g), Fr. 5-9 (16.5 g) and Fr. B5-10 (23.3 g).

Fr. B5-6 (36.2 g) was fractionated by reverse phase ODS column chromatography [400.0 g, MeOH:H₂O=(10:90)→(20:80)→(30:70)→(40:60)→(50:50)→(60:40)→(80:20)→MeOH] to give the following fractions: Fr. B5-6-1 (2646.4 mg), Fr. 5-6-2 (14178.8 mg), Fr. 5-6-3 (2071.5 mg), Fr. B5-6-4 (5135.8 mg), Fr. B5-6-5 (1126.6 mg), Fr. B5-6-6 (2503.7 mg), Fr. B5-6-7 (3700.5 mg), Fr. B5-6-8 (2089.0 mg), Fr. B5-6-9 (907.7 mg), Fr. B5-6-10 (336.8 mg), Fr. B5-6-11 (359.1 mg), Fr. B5-6-12 (545.1 mg), Fr. B5-6-13 (237.6 mg) and Fr. B5-6-14 (361.4 mg).

Fr. B5-6-4 (189.7 mg) was purified by separation by HPLC [column: COSMOSIL 5C₁₈-PAQ, eluent: CH₃CN:H₂O=(10:90)] to isolate inermioside A (compound 1, 18.9 mg, 0.00814%).

Fr. B5-6-8 (430.4 mg) was purified by separation by HPLC [column: COSMOSIL 5C₁₈-PAQ, eluent: CH₃CN:H₂O=(20:80)] to isolate apigenin 7-O-α-L-rhamnopyranosyl-(1→6)-β-D-glucopyranoside (compound 2, 10.1 mg, 0.00078%) and luteolin 4′-O-β-D-glucopyranoside (compound 3, 14.9 mg, 0.00115%).

The structural formulas of the isolated three compounds are shown below.

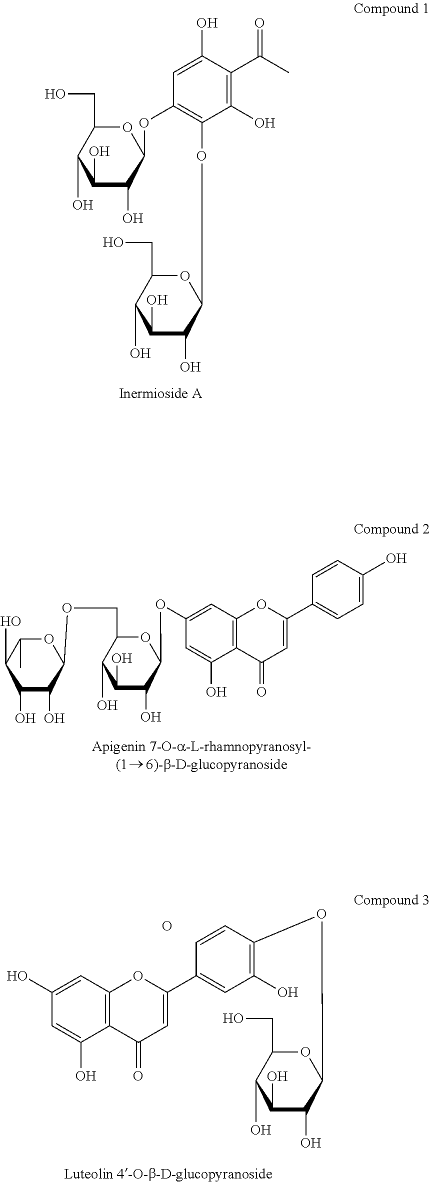

### Example 2: Anti-Malassezia Effect of Henna Leaf Extract and Compounds 1 to 3

### 1. Materials and Methods

### Culture of Malassezia

Malassezia used in this Example was a fungal strain (derived from scales from the scalp of a male human at the age of 40′s) possessed by NPR Medical Resource Laboratory. The fungal strain was identified to be Malassezia globosa by nucleotide sequence analysis of the D1/D2 region of 26S rDNA [601/601 (100%) matched]. The Malassezia strain was cultured in CHROMagar Malassezia/Candida prepared medium (Kanto Chemical Co., Ltd.).

### Preparation of YM Medium

In 1 L of distilled water were dissolved 10 g of D-glucose, 5 g of peptone, 3 g of yeast extract, 3 g of malt extract and 0.1 g of penicillin. The resulting solution was filtered and used for the experiments.

### Positive Control Drugs

As positive control drugs, streptomycin sulfate, amorolfine hydrochloride and itraconazole were used. Streptomycin sulfate is known as an antibiotic, but was also previously reported to show a fungistatic effect on yeasts (fungi) [Nature, 209, 536 (1966)]. Based on this report, streptomycin sulfate is employed as a positive control.

### Anti-Malassezia Tests

The henna leaf extract, compounds 1 to 3 and the positive control drugs were used as test substances for anti-Malassezia tests. A suspension of Malassezia was prepared to an optical density equivalent to McFarland's Standard No. 3 and 0.5 mL of the suspension was added to tubes. Each of the prepared samples, oleic acid and the YM medium were mixed into the tubes, and Malassezia was cultured at 37° C. for 22 hours. The fungi were stained using Microbial viability assay kit-WST (Dojindo Laboratories) and cultured at 37° C. for 2 hours. The absorbance was measured to calculate the viability of Malassezia. The results are expressed as a percentage (%) relative to a negative control (“control”: with no addition of test substances) taken as 100. A lower percentage indicates a stronger anti-Malassezia effect. The values obtained in the experiments were expressed as the mean±S.E. The significance of the difference in the means between the test substance administration groups and the negative control group was tested by the Dunnett's test. The significance level was set at 5% or 1%.

### 2. Results

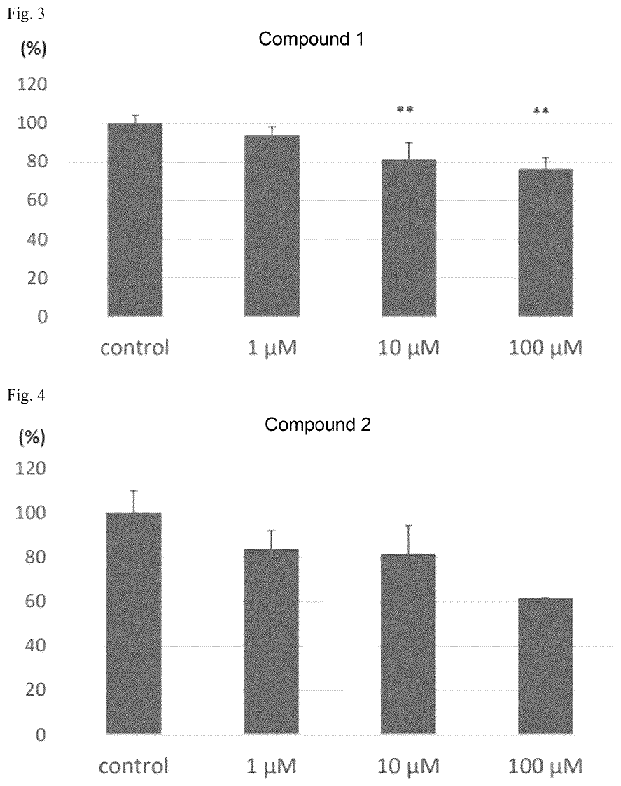

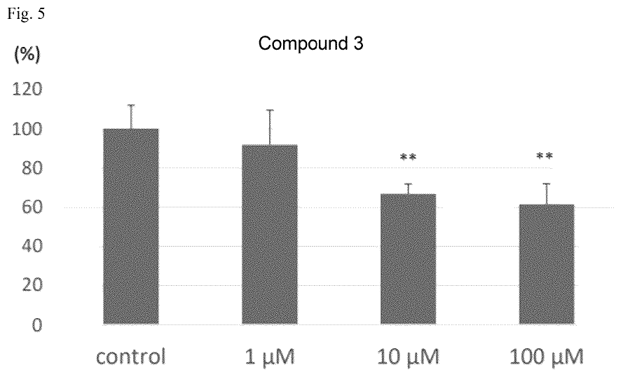

The results of the anti-Malassezia tests (the viability (%) of Malassezia) using the henna leaf extract and compounds 1 to 3 are shown in Tables 1 to 4 below and FIGS. 2 to 5 (n=4, in the figures, * represents a significant difference as compared to the negative control, *p<0.05, and **p<0.01).

TABLE 1
Henna leaf extract

| | Control | 0.5 mg/mL | 1 mg/mL |
|---|---|---|---|
| Mean | 100.0 | 85.3 | 75.6 |
| S.E. | 11.9 | 8.5 | 7.8 |

TABLE 2
Compound 1 Inermioside A

| | Control | 1 μM | 10 μM | 100 μM |
|---|---|---|---|---|
| Mean | 100.0 | 93.6 | 81.3 | 76.3 |
| S.E. | 4.1 | 4.2 | 8.7 | 5.6 |

TABLE 3
Compound 2 Apigenin 7-O-α-L-rhamnopyranosyl-
(1→6)-β-D-glucopyranoside

| | Control | 1 μM | 10 μM | 100 μM |
|---|---|---|---|---|
| Mean | 100.0 | 83.6 | 81.4 | 61.5 |
| S.E. | 9.9 | 8.3 | 12.7 | N.A. due to n = 2 |

TABLE 4
Compound 3 Luteolin 4′-O-β-D-glucopyranoside

| | Control | 1 μM | 10 μM | 100 μM |
|---|---|---|---|---|
| Mean | 100.0 | 91.8 | 66.7 | 61.3 |
| S.E. | 12.0 | 17.7 | 5.0 | 10.6 |

The inhibition of the viability of Malassezia by the henna leaf extract and compounds 1 to 3 became more prominent with increasing the amount added.

The results of the positive control drugs were as follows. When streptomycin sulfate is added at 10 μM, the viability of Malassezia was 105.9%, which indicates rather enhanced proliferation. A concentration of 137 μM or more is required to inhibit the viability by 20%. Amorolfine hydrochloride, which is an antifungal drug, is required to be at a concentration of 282.5 M to inhibit the viability of Malassezia by 20% or more. Itraconazole is required to be at a concentration of about 10 μM to inhibit the viability of Malassezia by about 20%.

The anti-Malassezia effect of compounds 1, 2 and 3 was comparable to or higher than that of the positive control drugs.

The present invention is not limited to each of the embodiments or Examples as described above, and various modifications are possible within the scope of the claims. Embodiments obtainable by appropriately combining the technical means disclosed in the different embodiments of the present invention are also included in the technical scope of the present invention. The contents of the scientific literature and the patent literature cited herein are hereby incorporated by reference in their entirety. CLAIMS

1. A method for inhibiting proliferation of Malassezia in a subject, comprising administering, to the subject, a compound (I) represented by the following formula (I): 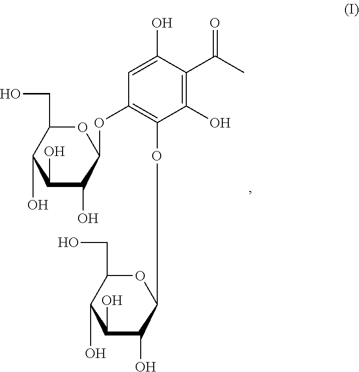
a compound (II) represented by the following formula (II): 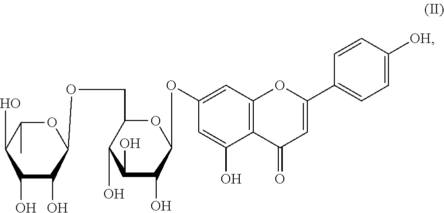
or
a compound (III) represented by the following formula (III): 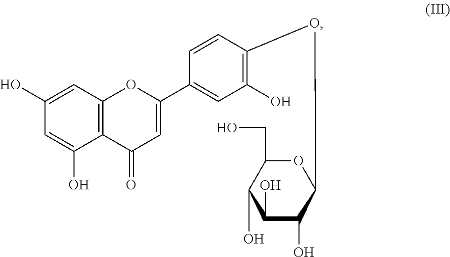
or
a pharmaceutically acceptable salt, ether derivative or ester derivative of the compound (I), the compound (II) or the compound (III).

2. The method according to claim 1, wherein the subject is a mammal.

3. The method according to claim 2, wherein the mammal is a human.

4. The method according to claim 1, wherein the administering is parenterally or orally administering.

5. The method according to claim 1, wherein the administering is application to a skin of the subject.

6. A method for inhibiting proliferation of Malassezia in a subject, comprising administering a henna extract to the subject.

7. The method according to claim 6, wherein the henna extract comprises at least one of compounds (I), (II) and (III) represented by the following formulas (I), (II) and (III): 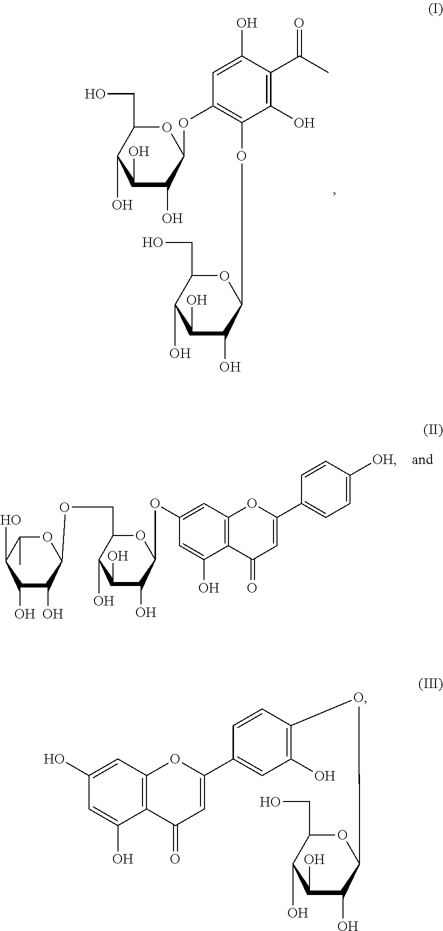
respectively, or a pharmaceutically acceptable salt thereof.

8. The method according to claim 6, wherein the henna extract is a henna leaf extract.

9. The method according to claim 6, wherein the subject is a mammal.

10. The method according to claim 9, wherein the mammal is a human.

11. The method according to claim 6, wherein the administering is parenterally or orally administering.

12. The method according to claim 6, wherein the administering is application to a skin of the subject.

13. A method for producing a compound (I) represented by the following formula (I): 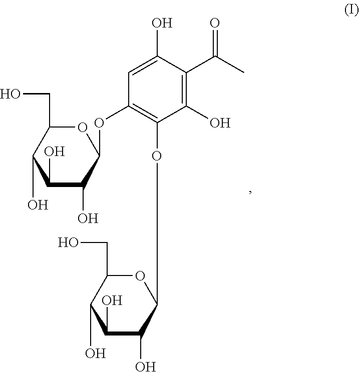
or
a pharmaceutically acceptable salt thereof, comprising
subjecting henna leaves to extraction to prepare an extract, and
obtaining the compound from the extract.
